(19) United States
(12) Patent Application Publication
(10) Pub. No.: US 2025/0049316 A1
(43) Pub. Date: Feb. 13, 2025

(54) OPTICAL BIOMETER

(71) Applicant: Crystalvue Medical Corporation, Taoyuan City (TW)

(72) Inventors: Yen-Jen CHANG, Taoyuan City (TW); Tung-Yu LEE, Taoyuan City (TW); Chun-Nan LIN, Taoyuan City (TW); Che-Liang TSAI, Taoyuan City (TW); Sung-Yang WEI, Taoyuan City (TW); Hsuan-Hao CHAO, Taoyuan City (TW); William WANG, Taoyuan City (TW); Ching Hung LIN, Taoyuan City (TW)

(21) Appl. No.: 18/792,960

(22) Filed: Aug. 2, 2024

Related U.S. Application Data
(60) Provisional application No. 63/531,373, filed on Aug. 8, 2023.

(51) Int. Cl. A61B 3/10

(52) U.S. Cl. CPC A61B 3/102

(57) ABSTRACT

An optical biometer includes a light-source module, a light-splitting module, a reference-arm, a sensing-arm and a sensing module. The light-source module emits incident-light. The light-splitting module, disposed corresponding to light-source module, divides the incident-light into reference light and sensing light. The reference-arm, disposed corresponding to light-splitting module, generates a first reflected-light according to the reference light. The sensing-arm, disposed corresponding to the light-splitting module, emits the sensing light to the eye and receives a second reflected-light from the eye. The sensing module generates a sensing result according to the first reflected-light and second reflected-light. In a first mode, the sensing light is emitted to a first position of the eye. In a second mode, the sensing light is emitted to a second position of the eye. The incident-light emitted by light-source module is partial annular light and the sensing result includes a partial annular image related to the eye.

### BACKGROUND OF THE INVENTION

### Cross Reference to Related Applications

This Application is a non-provisional application claiming priority to U.S. Provisional Application 63/531,373 filed on Aug. 8, 2023, which is incorporated by reference in its entirety.

### FIELD OF THE INVENTION

The invention relates to biometry; in particular, to an optical biometer.

### DESCRIPTION OF THE PRIOR ART

As shown in FIG. 1, an optical coherence tomography (OCT) biometer 1 includes a light source module LS, a light-splitting module SP, a reference arm RA, a sensing arm SA and a sensing module SE. An incident light LIN emitted by the light source module LS is divided into a reference light L1 and a sensing light L2 by the light-splitting module SP, and then the reference light L1 and the sensing light L2 are transmitted to the reference arm RA and the sensing arm SA respectively. The reference arm RA is used to reflect the reference light L1 to generate a first reflected light R1. The sensing arm SA is used to emit the sensing light L2 to the eye EYE and receive a second reflected light R2 from the eye EYE. The sensing module SE is used to receive the first reflected light R1 and the second reflected light R2 respectively and generate a sensing result according to the first reflected light R1 and the second reflected light R2. After analysis, the relative positions of interfaces in the eye are obtained, such as relative positions of retina and cornea.

As shown in FIG. 2A, the light source type of the conventional light source module LS can be a complete annular light source CRL disposed in front of the camera lens CM, which can emit a complete annular light to the eye EYE, so the camera lens CM receives the reflected light from the eye EYE and obtains a complete annular image IM1 as shown in FIG. 2B.

When the OCT biometer 1 measures the interfaces at different depths in the eye EYE, if the optical path length of the sensing arm SA is the same, the reference arm RA needs to have a larger optical path modulation range. In order to simultaneously cover the different optical path lengths required by the anterior chamber and fundus of the eye EYE, the reference arm EYE also needs to be able to quickly switch between different optical path lengths. However, the reference arm RA design used in the OCT biometer 1 cannot meet the above needs, further improvements are needed.

### SUMMARY OF THE INVENTION

Therefore, the invention provides an optical biometer to solve the above-mentioned problems of the prior arts.

A preferred embodiment of the invention is an optical biometer. In this embodiment, the optical biometer includes a light-source module, a light-splitting module, a reference arm, a sensing arm and a sensing module. The light-source module is configured to emit an incident-light. The light-splitting module is disposed corresponding to the light-source module and configured to divide the incident-light into a reference light and a sensing light. The reference arm is disposed corresponding to the light-splitting module and configured to generate a first reflected-light according to the reference light. The sensing arm is disposed corresponding to the light-splitting module and configured to emit the sensing light to an eye and receive a second reflected-light from the eye, wherein in a first mode, the sensing light is emitted to a first position of the eye; in a second mode, the sensing light is emitted to a second position of the eye. The sensing module is configured to generate a sensing result according to the first reflected-light and the second reflected-light, wherein the incident-light emitted by the light-source module is a partial annular light and the sensing result includes a partial annular image related to the eye.

In an embodiment, the first position of the eye is retina and the first mode is a retina mode.

In an embodiment, the second position of the eye is cornea and the second mode is a cornea mode.

In an embodiment, the reference arm includes a movable module, when the movable module is moved, an optical path of the first reflected light generated when the reference light is emitted to the movable module and reflected by the movable module changes accordingly.

In an embodiment, the light-splitting module is replaced by a switchable module.

In an embodiment, the light-source module includes a partial annular light source disposed in front of a camera lens.

In an embodiment, the light-source module includes a plurality of first light-emitting units and a plurality of second light-emitting units. The plurality of first light-emitting units is coupled to an annular light board and configured to emit the partial annular light. The plurality of second light-emitting units is disposed in an outer area and shared with other functional optical modules and configured to emit lights which is reflected by the mirror to an equivalent position.

In an embodiment, the light source module includes an annular light source, a lens module, an annular reflecting surface and a conical reflector. The conical reflector is disposed on the annular reflecting surface and the lens module is disposed between the annular light source and the conical reflector. An annular light emitted by the annular light source passes through the lens module and is reflected by the conical reflector to emit the incident light.

In an embodiment, the optical biometer is an optical coherence tomography (OCT) biometer and the optical biometer operates in spectral domain.

In an embodiment, the reference arm includes a movable reflector, a fixed reflector and a baffle. When the baffle operates in a first state, the reference light is only emitted to the fixed reflector. When the baffle operates in a second state, the reference light is only emitted to the movable reflector. When the baffle operates in a third state, the reference light is emitted to the fixed reflector and the movable reflector.

In an embodiment, in the first state, the baffle is moved to a first position corresponding to the movable reflector. In the second state, the baffle is moved to a second position corresponding to the fixed reflector. In the third state, the baffle is moved to a third position different from the first position and the second position.

Compared to the prior art, the optical biometer proposed by the invention can not only accurately measure the interfaces at different depths in the eye, but also provide a larger optical path modulation range and quickly switch between different optical path lengths at the same time.

The advantage and spirit of the invention may be understood by the following detailed descriptions together with the appended drawings.

### BRIEF DESCRIPTION OF THE APPENDED DRAWINGS

FIG. 1 illustrates a schematic diagram of an optical coherence tomography (OCT) biometer.

FIG. 2A and FIG. 2B respectively illustrate schematic diagrams of a complete annular light source disposed in front of camera lens and a complete annular image captured through the camera lens in the light source module in the prior art.

FIG. 3A and FIG. 3B respectively illustrate schematic diagrams of a partial annular light source disposed in front of camera lens and a partial annular image captured through the camera lens in a preferred embodiment of the invention.

FIG. 4A and FIG. 4B respectively illustrate a front view and a side view of the partial annular in front of the camera lens.

FIG. 5 illustrates a schematic diagram of a light source module in another embodiment of the invention.

FIG. 6 illustrates a schematic diagram of the reference arm in another embodiment of the invention.

FIG. 7A to FIG. 7C respectively illustrate schematic diagrams of the baffle of the reference arm operating in the first state to the third state in another embodiment of the invention.

### DETAILED DESCRIPTION OF THE INVENTION

Reference will now be made in detail to the exemplary embodiments of the present invention, examples of which are illustrated in the accompanying drawings. Elements/components with the same or similar numbers used in the drawings and embodiments are intended to represent the same or similar parts.

A specific embodiment of the invention is an optical biometer. In this embodiment, the optical biometer can be an optical coherence tomography (OCT) biometer and can operate in the spectral domain, but not limited to this.

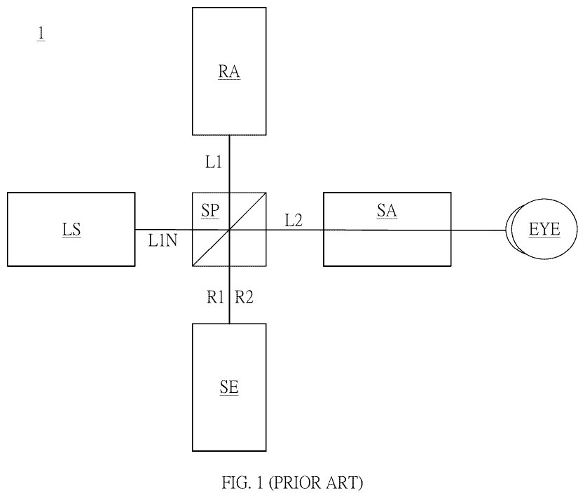

Please also refer to FIG. 1. The OCT biometer 1 of the invention also includes a light source module LS, a light-splitting module SP, a reference arm RA, a sensing arm SA and a sensing module SE. An incident light LIN emitted by the light source module LS is divided into a reference light L1 and a sensing light L2 by the light-splitting module SP, and then the reference light L1 is transmitted to the reference arm RA and the sensing light L2 is transmitted to the sensing arm SA respectively. The reference arm RA is configured to reflect the reference light L1 to generate a first reflected light R1. The sensing arm SA is configured to emit the sensing light L2 to the eye EYE and receive a second reflected light R2 from the eye EYE. The sensing module SE is configured to receive the first reflected light R1 and the second reflected light R2 respectively and generate a sensing result according to the first reflected light R1 and the second reflected light R2, and then obtain the relative positions of the interfaces in the eye EYE after analysis.

It should be noted that in a first mode, the sensing light L2 is emitted to the first position of the eye EYE, and in a second mode, the sensing light L2 is emitted to the second position of the eye EYE. The incident light LIN emitted by the light source module LS is a partial annular light and the sensing result includes a partial annular image related to the eye EYE. In fact, the first position of the eye EYE may be retina and the first mode may be a retina mode, and the second position of the eye EYE may be cornea and the second mode may be a cornea mode, but not limited to this.

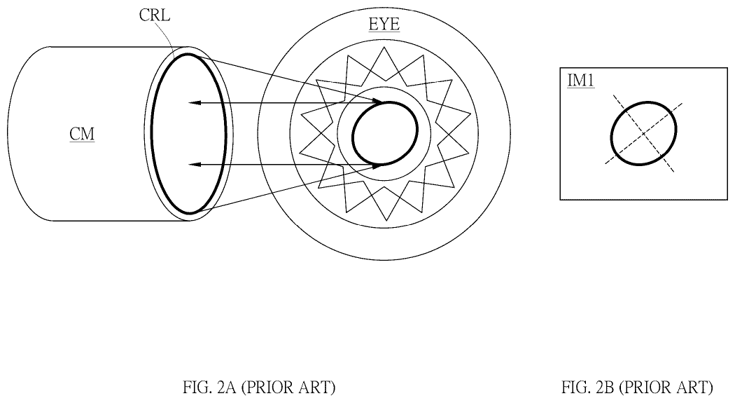

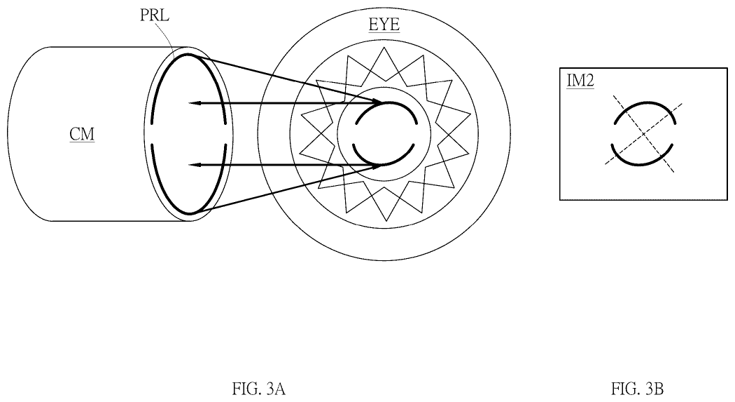

Please refer to FIG. 3A and FIG. 3B. FIG. 3A and FIG. 3B respectively illustrate schematic diagrams in which the light source module is a partial annular light source disposed in front of the camera lens and a partial annular image captured through the camera lens. Different from the light source type in FIG. 2A of the prior art that uses a complete annular light source CRL in front of the camera lens CM to emit a complete annular light to the eye EYE, in FIG. 3A of the invention, a partial annular light source is disposed in front of the camera lens CM to emit a partial annular light PRL to the eye EYE. Therefore, the invention can receive the reflected light of the eye EYE through the camera lens CM to obtain a partial annular image IM2 related to the eye EYE (as shown in FIG. 3B). It is obviously different from the camera lens CM in the prior art that receives the reflected light of the eye EYE and obtains a complete annular image IM1 related to the eye EYE (as shown in FIG. 2B).

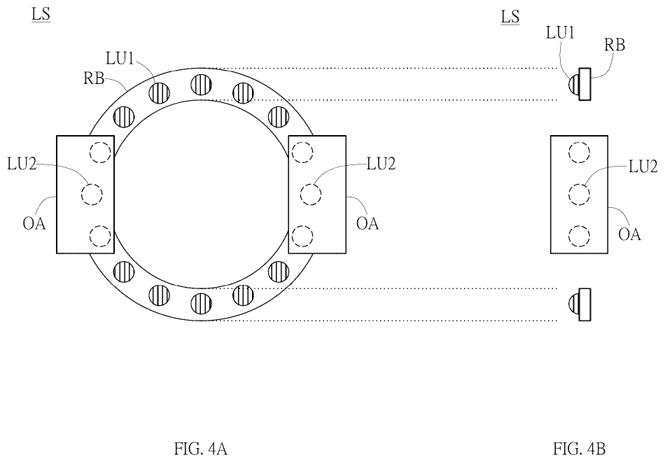

Please refer to FIG. 4A and FIG. 4B. FIG. 4A and FIG. 4B respectively illustrate a front view and a side view of a partial annular light source in front of the camera lens. As shown in FIG. 4A and FIG. 4B, the light source module LS includes a plurality of first light-emitting units LU1 and a plurality of second light-emitting units LU2. The plurality of first light-emitting units LU1 is coupled to an annular light board RB for emitting a partial annular light. The plurality of second light-emitting units LU2 is disposed in the outer area OA and can be shared with other functional optical modules, and their lights can be reflected to an equivalent position through a mirror. In fact, the actual placement position of the plurality of second light-emitting units LU2 can also be designed behind the annular light board RB or after multiple reflections, depending on its sharing with other functional optical modules.

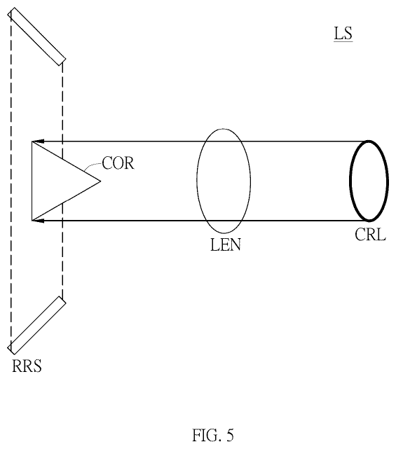

Please refer to FIG. 5, which is a schematic diagram of a light source module in another embodiment of the invention. As shown in FIG. 5, the light source module LS includes an annular light source CRL, a lens module LEN, an annular reflecting surface RRS, and a conical reflector COR. The conical reflector COR is disposed on the annular reflecting surface RRS, and the lens module LEN is disposed between the annular light source CRL and the conical reflector COR. The annular light emitted by the annular light source CRL passes through the lens module LEN and is reflected by the conical reflector COR disposed on the annular reflecting surface RRS to emit the incident light LIN.

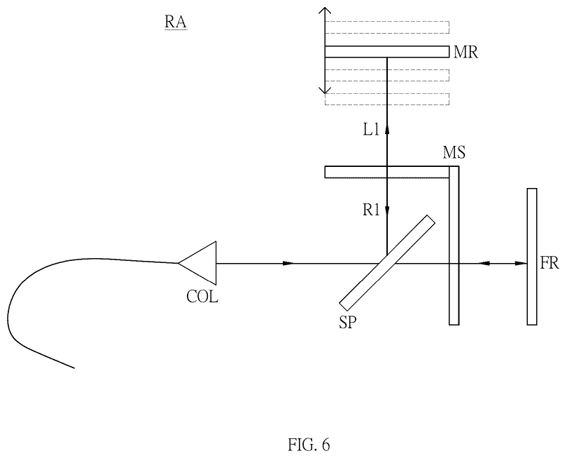

Please refer to FIG. 6, which is a schematic diagram of a reference arm in another embodiment of the invention. As shown in FIG. 6, the reference arm RA includes a collimator COL, a light-splitting module SP, a mechanical shutter MS, a movable mirror MR and a fixed mirror FR. In the second mode (e.g., the cornea mode), when the movable mirror MR is moved, the optical path of the first reflected light R1 generated by the reference light L1 emitted to the movable mirror MR and reflected by the movable mirror MR will also change accordingly. In practical applications, the light-splitting module SP in FIG. 6 can be replaced by a switchable module.

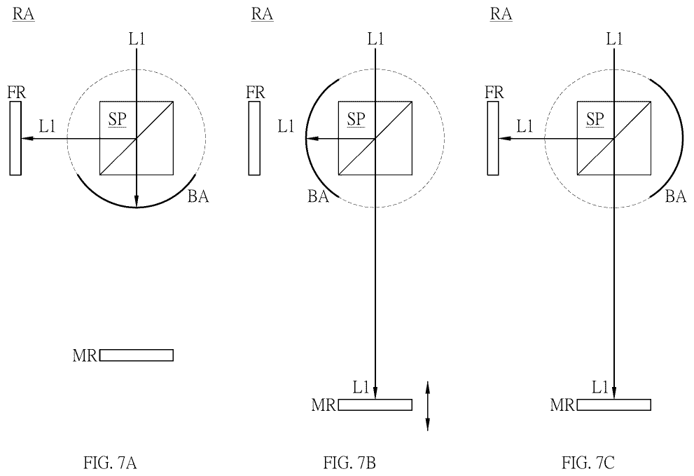

Please refer to FIG. 7A to FIG. 7C. FIG. 7A to FIG. 7C respectively illustrate schematic diagrams of the baffle of the reference arm operating in a first state to a third state in another embodiment of the invention.

In this embodiment, the optical coherent tomography (OCT) biometer operates in the spectrum domain, and its reference arm RA includes a movable mirror MR, a fixed mirror FR, a light-splitting module SP and a baffle BA. The baffle BA can operate in the first state to the third state shown in FIG. 7A to FIG. 7C respectively.

As shown in FIG. 7A, when the baffle BA operates in the first state, the baffle BA is moved to the first position corresponding to between the movable mirror MR and the light-splitting module SP. At this time, the baffle BA can block the reference light L1 emitted from the light-splitting module SP toward the movable reflector MR, so that the reference light L1 can be only emitted to the fixed reflector FR. In fact, after proper design, the first state of the baffle BA can be used to search for corneal signals. Under normal working conditions, the corneal signals can be located in the first ¼ to ⅓ area of the scanning line.

As shown in FIG. 7B, when the baffle BA operates in the second state, the baffle BA is moved to a second position between the fixed reflector FR and the light-splitting module SP. At this time, the baffle BA can block the reference light L1 emitted from the light-splitting module SP toward the fixed reflector FR, so that the reference light L1 can be only emitted to the movable reflector MR. In fact, after proper design, the second state of the baffle BA can be used to search for signals from other interfaces to be measured in the eye EYE, such as crystalline front/back surface signals or retinal signals. After finding other interface signals to be measured during the search process, the movable reflector MR allows the other interface signals to be measured to be located in the rear ½˜⅔ area of the scan line.

As shown in FIG. 7C, when the baffle BA operates in the third state, the baffle BA is moved to a third position, and the third position is different from the first position and the second position. At this time, since the baffle BA is not located at the first position between the movable reflector MR and the light-splitting module SP, nor located at the second position between the fixed reflector FR and the light-splitting module SP, so the baffle BA does not block the reference light L1 emitted by the light-splitting module SP toward the movable reflector MR and the fixed reflector FR. Therefore, the reference light L1 can be emitted to the fixed reflector FR and the movable reflector MR.

In fact, in the third state of the baffle BA, since the cornea signal and another interface signal to be measured (such as the retina signal) are located in the front and middle areas of the scan line respectively, they will not interfere with each other, so the reference light L1 can be transmitted to the fixed reflector FR and the movable reflector MR, so that the signals of the two interfaces to be measured can be measured at the same time and the distance between the two interfaces to be measured can be accurately measured.

It should be noted that the advantage of the operation mode of FIG. 7A to FIG. 7C is that there will be no time difference in the measured distance between the two interfaces to be measured in the eye, so the measurement accuracy can be effectively improved.

Compared to the prior art, the optical biometer proposed by the invention can not only accurately measure the interfaces at different depths in the eye, but also provide a larger optical path modulation range and quickly switch between different optical path lengths at the same time.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

What is claimed is:

1. An optical biometer, comprising:
a light-source module configured to emit an incident-light;
a light-splitting module, disposed corresponding to the light-source module and configured to divide the incident-light into a reference light and a sensing light;
a reference arm, disposed corresponding to the light-splitting module and configured to generate a first reflected-light according to the reference light;
a sensing arm, disposed corresponding to the light-splitting module and configured to emit the sensing light to an eye and receive a second reflected-light from the eye, wherein in a first mode, the sensing light is emitted to a first position of the eye; in a second mode, the sensing light is emitted to a second position of the eye; and
a sensing module configured to generate a sensing result according to the first reflected-light and the second reflected-light;
wherein the incident-light emitted by the light-source module is a partial annular light and the sensing result comprises a partial annular image related to the eye.

2. The optical biometer of claim 1, wherein the first position of the eye is retina and the first mode is a retina mode.

3. The optical biometer of claim 1, wherein the second position of the eye is cornea and the second mode is a cornea mode.

4. The optical biometer of claim 1, wherein the reference arm comprises a movable module, when the movable module is moved, an optical path of the first reflected light generated when the reference light is emitted to the movable module and reflected by the movable module changes accordingly.

5. The optical biometer of claim 1, wherein the light-splitting module is replaced by a switchable module.

6. The optical biometer of claim 1, wherein the light-source module comprises a partial annular light source disposed in front of a camera lens.

7. The optical biometer of claim 1, wherein the light-source module comprises:
a plurality of first light-emitting unit, coupled to an annular light board and configured to emit the partial annular light; and
a plurality of second light-emitting unit, disposed in an outer area and shared with other functional optical modules and configured to emit lights which is reflected by the mirror to an equivalent position.

8. The optical biometer of claim 1, wherein the light source module comprises an annular light source, a lens module, an annular reflecting surface and a conical reflector; the conical reflector is disposed on the annular reflecting surface and the lens module is disposed between the annular light source and the conical reflector, an annular light emitted by the annular light source passes through the lens module and is reflected by the conical reflector to emit the incident light.

9. The optical biometer of claim 1, wherein the optical biometer is an optical coherence tomography (OCT) biometer and the optical biometer operates in spectral domain.

10. The optical biometer of claim 9, wherein the reference arm comprises a movable reflector, a fixed reflector and a baffle; when the baffle operates in a first state, the reference light is only emitted to the fixed reflector; when the baffle operates in a second state, the reference light is only emitted to the movable reflector; when the baffle operates in a third state, the reference light is emitted to the fixed reflector and the movable reflector.

11. The optical biometer of claim 10, wherein in the first state, the baffle is moved to a first position corresponding to the movable reflector; in the second state, the baffle is moved to a second position corresponding to the fixed reflector; in the third state, the baffle is moved to a third position different from the first position and the second position.
